(19) United States
(12) Patent Application Publication
(10) Pub. No.: US 2021/0324454 A1
(43) Pub. Date: Oct. 21, 2021

(54) SYSTEMS AND METHODS FOR CORRECTING SAMPLE PREPARATION ARTIFACTS IN DROPLET-BASED SEQUENCING

(71) Applicant: 10x Genomics, Inc., Pleasanton, CA (US)

(72) Inventors: Preyas Shah, San Jose, CA (US); Li Wang, Pleasanton, CA (US); Brett Olsen, Alameda, CA (US)

(21) Appl. No.: 17/232,058

(22) Filed: Apr. 15, 2021

Related U.S. Application Data
(60) Provisional application No. 63/010,562, filed on Apr. 15, 2020.

(51) Int. Cl. C12Q 1/6816; C12Q 1/6858; C12Q 1/683

(52) U.S. Cl. CPC C12Q 1/6816; C12Q 1/683; C12Q 1/6858

(57) ABSTRACT

A method for filtering open chromatin regions on a cell barcode genomic sequence dataset is provided, comprising receiving, by one or more processors, a cell barcode genomic sequence dataset, the method comprising a plurality of fragment sequence reads and barcodes associated with the plurality of fragment sequence reads. The method further comprising generating, by the one or more processors, an adjacency matrix that counts up pairs of adjacent fragment sequence reads and barcodes associated with each fragment sequence read. The method further comprising identifying, by the one or more processors, pairs of adjacent fragment sequence reads with different barcodes and annotating the pair as a multiplet pair. The method further comprising filtering, by the one or more processors, one fragment sequence read from each of the identified multiplet pairs. The method further comprising generating, by the one or more processors, a multiplet filtered cell barcode genomic sequence dataset.

### CROSS REFERENCE

This application is related to U.S. Provisional Patent Application No. 63/010562, filed Apr. 15, 2020, entitled “Systems and methods for correcting sample preparation artifacts in droplet-based sequencing,” which is incorporated herein by reference in its entirety.

### FIELD

This description is generally directed towards systems and methods for identifying and correcting for droplet microfluidic errors in a cell barcode genomic sequence dataset. There is a need for improved detection and correction of artifacts arising during one or more steps of the multi-modal droplet-based single cell genomic sequencing technologies.

### BACKGROUND

Methods for probing genome-wide DNA accessibility have proven extremely effective in identifying regulatory elements across a variety of cell types and quantifying changes that lead to both activation or repression of gene expression. One such method is the Assay for Transposase Accessible Chromatin with high-throughput sequencing (ATAC-seq). The ATAC-seq method probes DNA accessibility with an artificial transposon, which inserts specific sequences into accessible regions of chromatin. Because the transposase can only insert sequences into accessible regions of chromatin not bound by transcription factors and/or nucleosomes, sequencing reads can be used to infer regions of increased chromatin accessibility.

Traditional approaches to the ATAC-seq methodology requires large pools of cells, processes cells in bulk, and result in data representative of an entire cell population, but lack information about cell-to-cell variation inherently present in a cell population (see, e.g., Buenrostro, et al., Curr. Protoc. Mol. Biol., 2015 Jan. 5; 21.29.1-21.29.9). While single cell ATAC-seq (scATAC-seq) methods have been developed and improve on traditional approaches by providing information about cell-to-cell variation inherently present in a cell population, these methods still suffer from limitations. One such limitation is the presence of multiplets in a sequencing data set, an issue inherent to the sequencing workflow that precedes scATAC-seq methods and any other single cell methods that employ barcoding as part of the library preparation steps in a sequencing workflow.

As such, there is a need with systems and methods that utilize multi-modal droplet-based single cell genomic sequencing technologies to be able to detect and correct for artifacts arising during one or more steps of the multi-modal droplet-based single cell genomic sequencing technologies.

### SUMMARY

In accordance with various embodiments, a method for filtering open chromatin regions on a cell barcode genomic sequence dataset is provided, comprising receiving, by one or more processors, a cell barcode genomic sequence dataset, the method comprising a plurality of fragment sequence reads and barcodes associated with the plurality of fragment sequence reads. The method further comprising generating, by the one or more processors, an adjacency matrix that counts up pairs of adjacent fragment sequence reads and barcodes associated with each fragment sequence read. The method further comprising identifying, by the one or more processors, pairs of adjacent fragment sequence reads with different barcodes and annotating the pair as a multiplet pair. The method further comprising filtering, by the one or more processors, one fragment sequence read from each of the identified multiplet pairs. The method further comprising generating, by the one or more processors, a multiplet filtered cell barcode genomic sequence dataset.

In accordance with various embodiments, there is provided a non-transitory computer-readable medium storing computer instructions for filtering open chromatin regions on a cell barcode genomic sequence dataset. The computer instructions comprising receiving, by one or more processors, a cell barcode genomic sequence dataset, the method comprising a plurality of fragment sequence reads and barcodes associated with the plurality of fragment sequence reads. The method further comprising generating, by the one or more processors, an adjacency matrix that counts up pairs of adjacent fragment sequence reads and barcodes associated with each fragment sequence read. The method further comprising identifying, by the one or more processors, pairs of adjacent fragment sequence reads with different barcodes and annotating the pair as a multiplet pair. The method further comprising filtering, by the one or more processors, one fragment sequence read from each of the identified multiplet pairs. The method further comprising generating, by the one or more processors, a multiplet filtered cell barcode genomic sequence dataset.

In accordance with various embodiments, there is provided a system for filtering open chromatin regions on a cell barcode genomric sequence dataset, comprising: a data source for receiving, by one or more processors, a cell barcode genomic sequence dataset comprising a plurality of fragment sequence reads and barcodes associated with the plurality of fragment sequence reads; a computing device communicatively connected to the data source and comprises: a matrix engine configured to generate an adjacency matrix that counts up pairs of adjacent fragment sequence reads and barcodes associated with each fragment sequence read; a pair identification engine configured to identify pairs of adjacent fragment sequence reads with different barcodes and annotating the pair as a multiplet pair; a filter engine configured to filter one fragment sequence read from each of the identified multiplet pairs; and an output engine configured to generate a multiplet filtered cell barcode genomic sequence dataset.

In accordance with various embodiments, a method for filtering open chromatin regions on a cell barcode genomic sequence dataset, is disclosed. One or more processors receive a cell barcode genomic sequence dataset comprising a plurality of fragment sequence reads and barcodes associated with the plurality of fragment sequence reads. The one or more processors generate an adjacency matrix that counts up pairs of adjacent fragment sequence reads and barcodes associated with each fragment sequence read. The one or more processors identify pairs of adjacent fragment sequence reads as a multiplet pair when each member of the pair of adjacent fragment sequence reads are found more often with different barcodes than with a same barcode. The one or more processors filters out one fragment sequence read from each of the identified multiplet pairs based on its associated barcode having a lowest count in the adjacency matrix. The one or more processors generates a multiplet filtered cell barcode genomic sequence dataset.

In accordance with various embodiments, a method for filtering open chromatin regions on a cell barcode genomic sequence dataset, is disclosed. One or more processors receive a cell barcode genomic sequence dataset comprising a plurality of fragment sequence reads and barcodes associated with the plurality of fragment sequence reads. The one or more processors generate an adjacency matrix that counts up pairs of adjacent fragment sequence reads and barcodes associated with each fragment sequence read. The one or more processors identify pairs of adjacent fragment sequence reads as a multiplet pair when each member of the pair of adjacent fragment sequence reads are found more often with different barcodes than with a same barcode. The one or more processors filters out one fragment sequence read from each of the identified multiplet pairs based on its associated barcode having more cross signal with another barcode than with the same associated barcode. The one or more processors generate a multiplet filtered cell barcode genomic sequence dataset.

These and other aspects and implementations are discussed in detail herein. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification.

### BRIEF DESCRIPTION OF FIGURES

The accompanying drawings are not intended to be drawn to scale. Like reference numbers and designations in the various drawings indicate like elements. For purposes of clarity, not every component may be labeled in every drawing. In the drawings:

FIG. 1 is a schematic illustration of a non-limiting example of the sequencing workflow for using single cell Assay for Transposase Accessible Chromatin (ATAC) sequencing to generate sequencing data for identifying genome-wide differential accessibility of gene regulatory elements, in accordance with various embodiments.

FIG. 2 is a schematic illustration of the production of adjacent fragments, in accordance with various embodiments.

FIG. 3 is an illustration of a fragment adjacency matrix for identifying multiplets, in accordance with various embodiments.

FIG. 4 is an illustration of multiplet types, in accordance with various embodiments.

FIG. 5 is an illustration of a fragment adjacency matrix for identifying multiplets, in accordance with various embodiments.

FIG. 6 is a schematic illustration of a non-limiting example of the workflow for filtering open chromatin regions on a cell barcode genomic sequence dataset, in accordance with various embodiments.

FIG. 7 is a schematic illustration of a non-limiting example of the workflow for filtering open chromatin regions on a cell barcode genomic sequence dataset, in accordance with various embodiments.

FIG. 8 is a schematic illustration of a non-limiting example of the workflow for filtering open chromatin regions on a cell barcode genomic sequence dataset, in accordance with various embodiments.

FIG. 9 is a schematic illustration of a non-limiting example of a system for filtering open chromatin regions on a cell barcode genomic sequence dataset, in accordance with various embodiments.

FIG. 10 is a block diagram that illustrates a computer system, upon which embodiments, or portions of the embodiments, may be implemented, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

This specification describes various exemplary embodiments of methods for conducting a customized analysis of open chromatin regions on a cell using a barcode genomic sequence dataset. It should be appreciated, however, that although the systems and methods disclosed herein refer to their application in open chromatin analysis specifically, they are equally applicable to other analogous fields at least from the perspective of the combination of initial analysis of a single data set, optional aggregation of a plurality of data sets, and customize analysis (or re-analysis) or the single or plural data sets around customized parameters.

In various embodiments, a computer program product can include instructions to receive an output file for a cell barcode genomic sequence dataset, the output file having various components such as a peak-barcode matrix and one or more cell clusters; instructions to adjusting one of more customizable parameters for analyzing the output file (e.g., peak barcode matrix), and instructions to generate an updated output file including an updated clustering of cells, based on the one or more customizable parameters, wherein each updated cell cluster includes cells with peaks representing a specific gene regulatory function.

In various embodiments, a system for conducting a customized analysis of open chromatin regions on a cell using a barcode genomic sequence dataset is provided and can include a data source for receiving, by one or more processors, an output file for the cell barcode genomic sequence dataset and one or more computing devices that can host and execute software code that comprises a clustering engine (and optionally a TF-barcode matrix engine and differential analysis engine). The clustering engine can be configured to generate an updated output file including updated clustering of cells, based on the one or more customizable parameters, wherein each updated cell cluster includes cells with peaks representing a specific gene regulatory function.

The disclosure, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion. In addition, as the terms “on,” “attached to,” “connected to,” “coupled to,” or similar words are used herein, one element (e.g., a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

It should be understood that any use of subheadings herein are for organizational purposes, and should not be read to limit the application of those subheaded features to the various embodiments herein. Each and every feature described herein is applicable and usable in all the various embodiments discussed herein and that all features described herein can be used in any contemplated combination, regardless of the specific example embodiments that are described herein. It should further be noted that exemplary description of specific features are used, largely for informational purposes, and not in any way to limit the design, subfeature, and functionality of the specifically described feature.

All publications mentioned herein are incorporated herein by reference for the purpose of describing and disclosing devices, compositions, formulations and methodologies which are described in the publication and which might be used in connection with the present disclosure.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one.

As used herein, the term “plurality” can be 2, 3, 4, 5, 6, 7, 8, 9, 10, or more.

As used herein, the terms “comprise”, “comprises”, “comprising”, “contain”, “contains”, “containing”, “have”, “having” “include”, “includes”, and “including” and their variants are not intended to be limiting, are inclusive or open-ended and do not exclude additional, unrecited additives, components, integers, elements or method steps. For example, a process, method, system, composition, kit, or apparatus that comprises a list of features is not necessarily limited only to those features but may include other features not expressly listed or inherent to such process, method, system, composition, kit, or apparatus.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, pharmacology and toxicology, cell and tissue culture, molecular biology, and protein and oligo- or polynucleotide chemistry and hybridization described herein are those available and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well-known and commonly used in the art.

DNA (deoxyribonucleic acid) is a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. Certain pairs of nucleotides specifically bind to one another in a complementary fashion (called complementary base pairing). That is, adenine (A) pairs with thymine (T) (in the case of RNA, however, adenine (A) pairs with uracil (U)), and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

The phrase “next generation sequencing” (NGS) refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization. More specifically, the MISEQ, HISEQ, NEXTSEQ, and NOVASEQ Systems of Illumina, the GRIDION and PROMETHION Systems of Oxford Nanopore Technologies, PACBIO SEQUEL Systems of Pacific Biosciences, and the Personal Genome Machine (PGM) and SOLiD Sequencing System of Life Technologies Corp, provide massively parallel sequencing of whole or targeted genomes. The SOLiD System and associated workflows, protocols, chemistries, etc. are described in more detail in PCT Publication No. WO 2006/084132, entitled “Reagents, Methods, and Libraries for Bead-Based Sequencing,” international filing date Feb. 1, 2006, U.S. patent application Ser. No. 12/873,190, entitled “Low-Volume Sequencing System and Method of Use,” filed on Aug. 31, 2010, and U.S. patent application Ser. No. 12/873,132, entitled “Fast-Indexing Filter Wheel and Method of Use,” filed on Aug. 31, 2010, the entirety of each of these applications being incorporated herein by reference thereto.

The phrase “sequencing run” refers to any step or portion of a sequencing experiment performed to determine some information relating to at least one biomolecule (e.g., nucleic acid molecule).

The term “genome,” as used herein, generally refers to genomic information from a subject, which may be, for example, at least a portion or an entirety of a subject's hereditary information. A genome can comprise coding regions (e.g., that code for proteins) as well as non-coding regions. A genome can include the sequence of all chromosomes together in an organism. For example, the human genome ordinarily has a total of 46 chromosomes. The sequence of all of these together may constitute a human genome.

As used herein, the phrase “genomic features” can refer to a genome region with some annotated function (e.g., a gene, protein coding sequence, mRNA, tRNA, rRNA, repeat sequence, inverted repeat, miRNA, siRNA, etc.) or a genetic/genomic variant (e.g., single nucleotide polymorphism/variant, insertion/deletion sequence, copy number variation, inversion, etc.) which denotes a single or a grouping of genes (in DNA or RNA) that have undergone changes as referenced against a particular species or sub-populations within a particular species due to mutations, recombination/crossover or genetic drift.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject.

In some examples, such systems provide “sequencing reads” (also referred to as “fragment sequence reads” or “reads” herein). A read may include a string of nucleic acid bases corresponding to a sequence of a nucleic acid molecule that has been sequenced. In some situations, systems and methods provided herein may be used with proteomic information.

In general, the methods and systems described herein accomplish targeted genomic sequencing by providing for the determination of the sequence of long individual nucleic acid molecules and/or the identification of direct molecular linkage as between two sequence segments separated by long stretches of sequence, which permit the identification and use of long range sequence information, but this sequencing information is obtained using methods that have the advantages of the extremely low sequencing error rates and high throughput of short read sequencing technologies. The methods and systems described herein segment long nucleic acid molecules into smaller fragments that can be sequenced using high-throughput, higher accuracy short-read sequencing technologies, and that segmentation is accomplished in a manner that allows the sequence information derived from the smaller fragments to retain the original long range molecular sequence context, i.e., allowing the attribution of shorter sequence reads to originating longer individual nucleic acid molecules. By attributing sequence reads to an originating longer nucleic acid molecule, one can gain significant characterization information for that longer nucleic acid sequence that one cannot generally obtain from short sequence reads alone. This long range molecular context is not only preserved through a sequencing process, but is also preserved through the targeted enrichment process used in targeted sequencing approaches described herein, where no other sequencing approach has shown this ability.

In general, sequence information from smaller fragments will retain the original long range molecular sequence context through the use of a tagging procedure, including the addition of barcodes as described herein and known in the art. In specific examples, fragments originating from the same original longer individual nucleic acid molecule will be tagged with a common barcode, such that any later sequence reads from those fragments can be attributed to that originating longer individual nucleic acid molecule. Such barcodes can be added using any method known in the art, including addition of barcode sequences during amplification methods that amplify segments of the individual nucleic acid molecules as well as insertion of barcodes into the original individual nucleic acid molecules using transposons, including methods such as those described in Amini et al., Nature Genetics 46: 1343-1349 (2014) (advance online publication on Oct. 29, 2014), which is hereby incorporated by reference in its entirety for all purposes and in particular for all teachings related to adding adaptor and other oligonucleotides using transposons. Once nucleic acids have been tagged using such methods, the resultant tagged fragments can be enriched using methods described herein such that the population of fragments represents targeted regions of the genome. As such, sequence reads from that population allows for targeted sequencing of select regions of the genome, and those sequence reads can also be attributed to the originating nucleic acid molecules, thus preserving the original long range molecular sequence context. The sequence reads can be obtained using any sequencing methods and platforms known in the art and described herein.

In addition to providing the ability to obtain sequence information from targeted regions of the genome, the methods and systems described herein can also provide other characterizations of genomic material, including without limitation haplotype phasing, identification of structural variations, and identifying copy number variations, as described in co-pending applications U.S. Ser. Nos. 14/752,589 and 14/752,602, both filed on Jun. 26, 2015), which are herein incorporated by reference in their entirety for all purposes and in particular for all written description, figures and working examples directed to characterization of genomic material.

Methods of processing and sequencing nucleic acids in accordance with the methods and systems described in the present application are also described in further detail in U.S. Ser. Nos. 14/316,383; 14/316,398; 14/316,416; 14/316,431; 14/316,447; and 14/316,463 which are herein incorporated by reference in their entirety for all purposes and in particular for all written description, figures and working examples directed to processing nucleic acids and sequencing and other characterizations of genomic material.

The term “barcode,” as used herein, generally refers to a label, or identifier, that conveys or is capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include barcode sequences, such as: polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing reads.

The terms “adaptor(s)”, “adapter(s)” and “tag(s)” may be used synonymously. An adaptor or tag can be coupled to a polynucleotide sequence to be “tagged” by any approach, including ligation, hybridization, or other approaches. In various embodiments within the disclosure, the term adapter can refer to customized strands of nucleic acid base pairs created to bind with specific nucleic acid sequences, e.g., sequences of DNA.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

The term “macromolecule” or “macromolecular constituent,” as used herein, generally refers to a macromolecule contained within or from a biological particle. The macromolecular constituent may comprise a nucleic acid. In some cases, the biological particle may be a macromolecule. The macromolecular constituent may comprise DNA. The macromolecular constituent may comprise RNA. The RNA may be coding or non-coding. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

The term “partition,” as used herein, generally, refers to a space or volume that may be suitable to contain one or more species or conduct one or more reactions. A partition may be a physical compartment, such as a droplet or well. The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses).

The term “sample,” as used herein, generally refers to a “biological sample” of a subject. The sample may be obtained from a tissue of a subject. The sample may be a cell sample. A cell may be a live cell. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears. In some embodiments, the term “sample” can refer to a cell or nuclei suspension extracted from a single biological source (blood, tissue, etc.).

The sample may comprise any number of macromolecules, for example, cellular macromolecules. The sample maybe or may include one or more constituents of a cell, but may not include other constituents of the cell. An example of such cellular constituents is a nucleus or an organelle. The sample may be or may include DNA, RNA, organelles, proteins, or any combination thereof. The sample may be or include a chromosome or other portion of a genome. The sample may be or may include a bead (e.g., a gel bead) comprising a cell or one or more constituents from a cell, such as DNA, RNA, nucleus, organelles, proteins, or any combination thereof, from the cell. The sample may be or may include a matrix (e.g., a gel or polymer matrix) comprising a cell or one or more constituents from a cell, such as DNA, RNA, nucleus, organelles, proteins, or any combination thereof, from the cell.

As used herein, the term “fragment” refers to unique ATAC-seq fragment captured by the ATAC-seq assay. Each fragment can be created by two separate transposition events, which create the two ends of the observed fragment. Each unique fragment may generate multiple duplicate reads. These duplicate reads can be collapsed into a single fragment record.

In some embodiments, the term “fragment” can also refer to a piece of genomic DNA, bound by two adjacent cut sites, that has been converted into a sequencer-compatible molecule with an attached cell-barcode. The alignment interval of such fragment can be obtained by correcting the alignment interval of the sequenced fragment by +4 base-pair (bp) on the left end of the fragment, and −5 bp on the right end (where left and right are relative to genomic coordinates). It is understood that, such correction is performed to account for the 9 bp of DNA that the transposase occupies when it cuts the DNA (accessibility of chromatin is recorded around the center of this 9 bp stretch). Most fragment-based metrics computed by the analysis workflow can be based on fragments that passed various quality filters.

As used herein, the term “nucleosome” refers to structural units of the DNA formed by histones that help package the eukaryotic DNA into well-organized chromosomes.

As used herein, the term “histone” refers to protein found in eukaryotic cell nuclei that forms nucleosomes.

As used herein, the term “cell barcode” refers to any barcodes that have been determined to be associated with a cell.

As used herein, the term “Gel bead-in-EMulsion” or “GEM” refers to a droplet containing some sample volume and a barcoded gel bead, forming an isolated reaction volume. When referring to the subset of the sample contained in the droplet, the term “partition” may also be used. In various embodiments within the disclosure, the term barcode can refer to a GEM containing a gel bead that carries many DNA oligonucleotides with the same barcode, whereas different GEMs have different barcodes.

As used herein, the term “EM well” or “GEM group” refers to a set of partitioned cells (Gel beads-in-EMulsion or GEMs) from a single 10x Chromium™ Chip channel. One or more sequencing libraries can be derived from a GEM well.

As used herein, the term “PCR duplicates” refers to duplicates created during PCR amplification. During PCR amplification of the fragments, each unique fragment that is created may result in multiple read-pairs sequenced with near identical barcodes and sequence data. These duplicate reads are identified computationally, and collapsed into a single fragment record for downstream analysis.

As used herein, the term “peak” refers to a compact region of the genome identified as having “open chromatin” due to an enrichment of cut-sites inside the region.

As used herein, the term “promoter” refers to a region of DNA that initiates transcription of a particular gene. Promoters can be located near the transcription start sites of genes, on the same strand and upstream on the DNA.

As used herein, the term “read data” refers to raw genomic data from sequenced DNA.

As used herein, the term “read-pair” refers to the read data sequenced from one molecule. This can include read1, read2, and the barcode sequence read.

As used herein, the term “sequencing run” refers to a flowcell containing data from one sequencing instrument run. The sequencing data can be further addressed by lane and by one or more sample indices.

As used herein, the “targeted region” refers to any known, annotated, epigenetically relevant regions in the genome such as transcription start sites (TSS), enhancers, promoters or DNase hypersensitive sites. The embodiment metrics often refer to these as targeted regions.

As used herein, the term “transcription Factor (TF)” refers to a protein that controls the rate of transcription of genetic information from DNA to messenger RNA, by binding to a specific DNA sequences (like promoter or enhancers) that are commonly located in the vicinity of the gene they control.

As used herein, the term “transposase enzyme” refers to an enzyme that can cut open chromatin and ligate adapters to the 3′ end of each DNA strand.

As used herein, the term “cut site” or “cut-site” refers to location on the genome where transposase cuts the DNA and inserts adapters.

As used herein, the term “transposition” refers to the reaction carried out by the transposase enzyme.

As used herein, the term “transcription start site” or “TSS” is the location where transcription starts at the 5′-end of a gene sequence.

### Single Cell ATAC Sequencing Worklfow

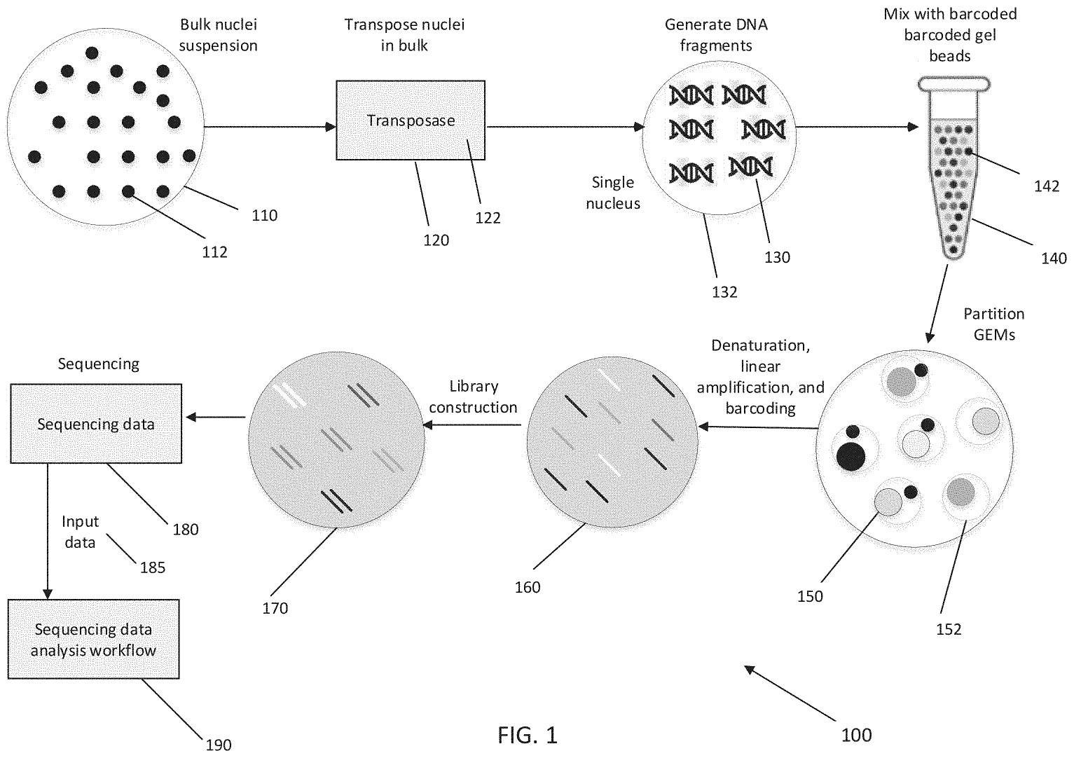

In accordance with various embodiments, a general schematic workflow is provided in FIG. 1 to illustrate a non-limiting example process for using single cell Assay for Transposase Accessible Chromatin (ATAC) sequencing technology to generate sequencing data. Such sequencing data can be used for identifying genome-wide differential accessibility of gene regulatory elements in accordance with various embodiments. The workflow can include various combinations of features, whether it be more or less features than that illustrated in FIG. 1. As such, FIG. 1 simply illustrates one example of a possible workflow.

### Nuclei Isolation

FIG. 1 provides a schematic workflow 100, the workflow including a bulk nuclei suspension 110 from a sample comprising a plurality of individual nuclei 112. In various embodiments, obtaining a bulk nuclei suspension can include isolating nuclei in bulk from a sample. It is understood that one problem with generating ATAC sequencing datasets, is that the dataset may contain a large percentage of read sequences (also referred to as reads) from mitochondrial DNA. Various methods, in accordance with various embodiments herein, can be employed for ensuring low mitochondrial reads from samples and high quality nuclei sequencing data. Accordingly, in some embodiments, preparation of the bulk nuclei suspension can include carefully extracting nuclei from cells, while ensuring the mitochondria stays intact. Various known protocols can be employed to isolate, wash, count nuclei, and generate nuclei suspensions for use with the single cell ATAC sequencing protocol of the embodiments herein. Nuclei for generating the nuclei suspensions can be isolated from any cells. Such cells may include, but are not limited to, cells from fresh and cryopreserved cell lines, e.g., human and mouse cell lines, as well as more fragile primary cells. In various embodiments, such cells may include, any eukaryotic cells, i.e., a eukaryotic cell with a chromatin structure. In various embodiments, such cells may include, but are not limited to, immune cells (e.g., B cells and T cells), peripheral blood mononuclear cells (PBMCs), Bone Marrow Mononuclear Cells (BMMCs), skin cells, cancer cells, embryonic neurons, and adult neurons. In various embodiments, nuclei for generating the nuclei suspensions can be isolated from different human and mouse tissues. In various embodiments, nuclei for generating the nuclei suspensions can be isolated from different human and mouse tumor samples.

### Transpose Nuclei in Bulk and Generate DNA Fragments

The workflow 100 provided in FIG. 1 further includes transposing the bulk nuclei suspension and generating adapter-tagged DNA fragments. The bulk nuclei suspension 110 is incubated with a transposition mix 120 containing Transposase 122. Upon incubation, the Transposase 122 enters individual nuclei 112 and preferentially fragments the DNA in open regions of a chromatin to generate a plurality of adapter-tagged DNA fragments 130 inside individual transposed nucleus 132.

In various embodiments, transposing the bulk nuclei suspensions can include incubating the nuclei suspension with a transposition mix that includes a Transposase enzyme, e.g., a Tn5 transposase. The transposase can be a mutated, hyperactive Tn5 transposase. In some embodiments, the transposase can be a Mu transposase. The transposase enters the nuclei and preferentially fragments the DNA in open regions of the chromatin by a process called transposing. More specifically, in various embodiments herein, the process results in transposing the nuclei in a bulk solution. Simultaneously during this process, adapter sequences can be added to the ends of the DNA fragments by the transposase. This process results in adapter-tagged DNA fragments inside individually transposed nucleus.

### GEM Generation

The workflow 100 provided in FIG. 1 further includes Gel beads-in-EMulsion (GEMs) generation. With the adapter-tagged DNA fragments 130 in hand, the bulk nuclei suspension containing the individual transposed nucleus 132 is mixed with a gel beads solution 140 containing a plurality of individually barcoded gel beads 142. In various embodiments, this step results in partitioning the nuclei into a plurality of individual GEMs 150, each including a single transposed nucleus 132 that contains a plurality of adapter-tagged DNA fragments 110, and a barcoded gel bead 142. This step also results in a plurality of GEMs 152, each containing a barcoded gel bead 142 but no nuclei. Detail related to GEM generation, in accordance with various embodiments disclosed herein, is provided below.

In various embodiments, GEMs can be generated by combining barcoded gel beads, transposed nuclei containing the transposase adapter-tagged DNA fragments, and other reagents or a combination of biochemical reagents that may be necessary for the GEM generation process. Such reagents may include, but are not limited to, a combination of biochemical reagents (e.g., a master mix) suitable for GEM generation and partitioning oil. The barcoded gel beads 142 of the various embodiments herein may include a gel bead attached to oligonucleotides containing (i) an Illumina® P5 sequence (adapter sequence), (ii) a 16 nucleotide (nt) 10x Barcode, and (iii) a Read 1 (Read 1N) sequencing primer sequence. It is understood that other adapter, barcode, and sequencing primer sequences can be contemplated within the various embodiments herein.

In various embodiments, GEMs are generated by partitioning the transposed nuclei (containing the transposase adapter-tagged DNA fragments) using a microfluidic chip. The microfluidic chip of the various embodiments herein can be a Chromium Chip E. To achieve single nuclei resolution per GEM, the nuclei can be delivered at a limiting dilution, such that the majority (e.g., ˜90-99%) of the generated GEMs do not contains any nuclei, while the remainder of the generated GEMs largely contain a single nucleus.

### Barcoding DNA Fragments

The workflow 100 provided in FIG. 1 further includes barcoding the adapter-tagged DNA fragments 130 for producing a plurality of uniquely barcoded single-stranded DNA fragments 160. Upon generation of the GEMs 150, the gel beads 142 can be dissolved releasing the various oligonucleotides of the embodiments described above, which are then mixed with the adapter-tagged DNA fragments 130 resulting in a plurality of uniquely barcoded single-stranded DNA fragments 160 following amplification of the GEMs 150. Detail related to generation of the plurality of uniquely barcoded single-stranded DNA fragments 160, in accordance with various embodiments disclosed herein, is provided below.

In various embodiments, upon generation of the GEMs 150, the gel beads 142 can be dissolved, and oligonucleotides of the various embodiments disclosed herein, containing the Illumina® P5 sequence (adapter sequence), an unique 10x Barcode, and Read 1 sequencing primer sequence can be released and mixed with the adapter-tagged DNA fragment and other reagents or a combination of biochemical reagents (e.g., a master mix necessary for the amplification process). Methods such as denaturation and linear amplification during thermal cycling of the GEMs or splinted ligation can then be performed to produce a plurality of uniquely barcoded single-stranded DNA fragments 160. In various embodiments herein, the plurality of uniquely barcoded single-stranded DNA fragments 160 can be 10x barcoded single-stranded DNA fragments. In one non-limiting example of the various embodiments herein, a pool of ˜750,000, 10x barcodes are utilized to uniquely index and barcode the transposed DNA fragments of each individual nucleus.

Accordingly, barcoded products of the various embodiments herein can include a plurality of 10x barcoded single-stranded DNA fragments generated during the thermal cycling process. In one non-limiting example of the various embodiments herein, each such 10x barcoded single-stranded DNA fragment can include a Illumina® P5 sequence (adapter sequence), a unique 10x barcode, a Read 1 sequencing primer sequence, a transposase adapter-tagged DNA fragment or insert, and a Read 2 (Read 2N)) sequencing primer sequence.

In various embodiments, after the amplification and barcoding process, the GEMs 150 are broken and pooled DNA fractions are recovered. The adapter-flanked, 10x barcoded DNA fragments are released from the droplets, i.e., the GEMs 150, and processed in bulk to complete library preparation for sequencing (e.g., next generation high throughput sequencing such as the single cell ATAC sequencing), as described in detail below. In various embodiments, following the amplification process, leftover biochemical reagents can be removed from the post-GEM reaction mixture. In one embodiment of the disclosure, silane magnetic beads can be used to remove leftover biochemical reagents. Additionally, in accordance with embodiments herein, the unused barcodes from the sample can be eliminated, for example, by Solid Phase Reversible Immobilization (SPRI) beads.

### Library Construction

The workflow 100 provided in FIG. 1 further includes a library construction step. In the library construction step of workflow 100, a library 170 containing a plurality of double-stranded DNA fragments are generated. These double-stranded DNA fragments can be utilized for completing the subsequent sequencing step, e.g., the single cell ATAC sequencing step. Detail related to the library construction, in accordance with various embodiments disclosed herein, is provided below.

In accordance with various embodiments disclosed herein, an Illumina® P7 sequence (adapter sequence) and a sample index (SI) sequence (e.g., i7) can be added during the library construction step via PCR to generate the library 170, which contains a plurality of double stranded DNA fragments. In accordance with various embodiments herein, the sample index sequences can each comprise of one or more oligonucleotides. In one embodiment, the sample index sequences can each comprise of four oligonucleotides. In various embodiments, when analyzing the single cell ATAC sequencing data for a given sample, the reads associated with all four of the oligonucleotides in the sample index can be combined for identification of a sample. Accordingly, in one non-limiting example, the final single cell ATAC sequencing libraries contain sequencer compatible double-stranded DNA fragments containing the P5 and P7 sequences used in Illumina® bridge amplification, a unique 10x barcode sequence, and Read 1 and Read 2 sequencing primer sequences.

Various embodiments of single cell ATAC sequencing technology within the disclosure can at least include platforms such as One Sample, One GEM Well, One Flowcell; One Sample, One GEM well, Multiple Flowcells; One Sample, Multiple GEM Wells, One Flowcell; Multiple Samples, Multiple GEM Wells, One Flowcell platform; and Multiple Samples, Multiple GEM Wells, One Flowcell. Accordingly, various embodiments within the disclosure can include sequence dataset from one or more samples, samples from one or more donors, and multiple libraries from one or more donors.

### Sequencing

The workflow 100 provided in FIG. 1 further includes a sequencing step. In this step, the library 170 can be sequenced to generate a plurality of sequencing data 180. The fully constructed library 170 can be sequenced according to a suitable sequencing technology, such as a next-generation sequencing protocol, to generate the sequencing data 180. In various embodiments, the next-generation sequencing protocol utilizes the llumina® sequencer for generating the sequencing data. It is understood that other next-generation sequencing protocols, platforms, and sequencers such as, e.g., MiSeq™, NextSeg· 500/550 (High Output), HiSeq 2500™ (Rapid Run), HiSeg™ 3000/4000, and NovaSeg™, can be also used with various embodiments herein.

### Sequencing Data Input and Data Analysis Workflow

The workflow 100 provided in FIG. 1 further includes a sequencing data analysis workflow 190. With the sequencing data 180 in hand, the data can then be output, as desired, and used as an input data 185 for the downstream sequencing data analysis workflow 190 for identifying differential accessibility of gene regulatory elements in transposase accessible open chromatin regions, in accordance with various embodiments herein. Sequencing the single cell ATAC libraries produces standard output sequences (also referred to as the “single cell ATAC sequencing data”, “sequencing data”, “sequence data”, or the “sequence output data”) that can then be used as the input data 185, in accordance with various embodiments herein. The sequence data contains sequenced fragments (also interchangeably referred to as “fragment sequence reads”, “sequencing reads” or “reads”), which in various embodiments include DNA sequences of the transposase adapter-tagged fragments containing the associated 10x barcode sequences, adapter sequences, and primer oligo sequences.

The various embodiments, systems and methods within the disclosure further include processing and inputting the sequence data. A compatible format of the sequencing data of the various embodiments herein can be a FASTQ file. Other file formats for inputting the sequence data is also contemplated within the disclosure herein. Various software tools within the embodiments herein can be employed for processing and inputting the sequencing output data into input files for the downstream data analysis workflow. One example of a software tool that can process and input the sequencing data for downstream data analysis workflow is the cellranger-atac mkfastq tool within the Cell Ranger™ ATAC analysis pipeline. It is understood that, various systems and methods with the embodiments herein are contemplated that can be employed to independently analyze the inputted single cell ATAC sequencing data for identifying genome-wide differential accessibility of gene regulatory elements in accordance with various embodiments.

### Detection and Correction of Gel Bead Artifacts

As stated above, in various embodiments, transposing bulk nuclei suspensions can include incubating the nuclei suspension with a transposition mix that includes a Transposase enzyme, e.g., a Tn5 transposase. In some embodiments, the transposase can be a mutated, hyperactive Tn5 transposase. In some embodiments, the transposase can be a Mu transposase. The transposase enters the nuclei and preferentially fragments the DNA in open regions of the chromatin by a process called transposing. More specifically, in various embodiments herein, the process results in transposing the nuclei in a bulk solution. Simultaneously during this process, adapter sequences can be added to the ends of the DNA fragments by the transposase. This process results in adapter-tagged DNA fragments inside an individually transposed nucleus.

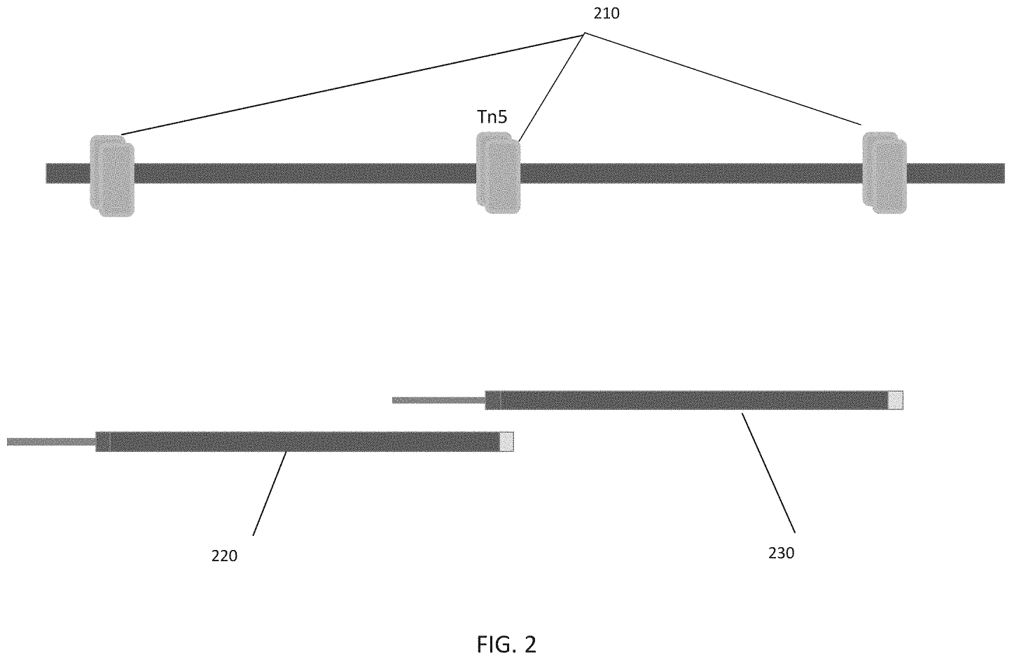

As stated above, Tn5 transposase tagments the free DNA, producing many small fragments with adapters on either end. Fragments can be sequenced if two Tn5 enzymes cut at close (<1 kb) locations in the same orientation, so the fragment between them has the correct set of adapters. Therefore, if three Tn5 enzymes cut at nearby locations in the same orientation, two fragments are produced that share a cut site between them. Each of those fragments are then barcoded independently by whatever barcodes are available in the GEM. This is illustrated in FIG. 2, where a transposase 210 fragments the DNA at sites that produce directly adjacent fragments 220 and 230, which generally would and should possess identical barcodes. However, in certain circumstances, largely due to barcoding-related errors during bead manufacturing or the sample preparation process, as illustrated in FIG. 1, those adjacent fragments 220 and 230 can be tagged with different barcodes. Therefore, rather than producing a signal that would normally be from one barcode common to both fragments, the signal is incorrectly split between two different barcodes, causing inaccuracies in the data and inaccuracies in the downstream computational analysis. These pairs of fragments from a common cell but with different barcodes are generally be referred to as multiplets. As discussed above, multiplets can be formed under two typical scenarios. Multiplets can be formed when GEMs contain two different barcode beads and a single cell, which is referred to as gel-bead multiplets. Multiplets can also be formed when GEMs contain a single bead that is contain different barcode sequences, which is referred to as barcode multiplets.

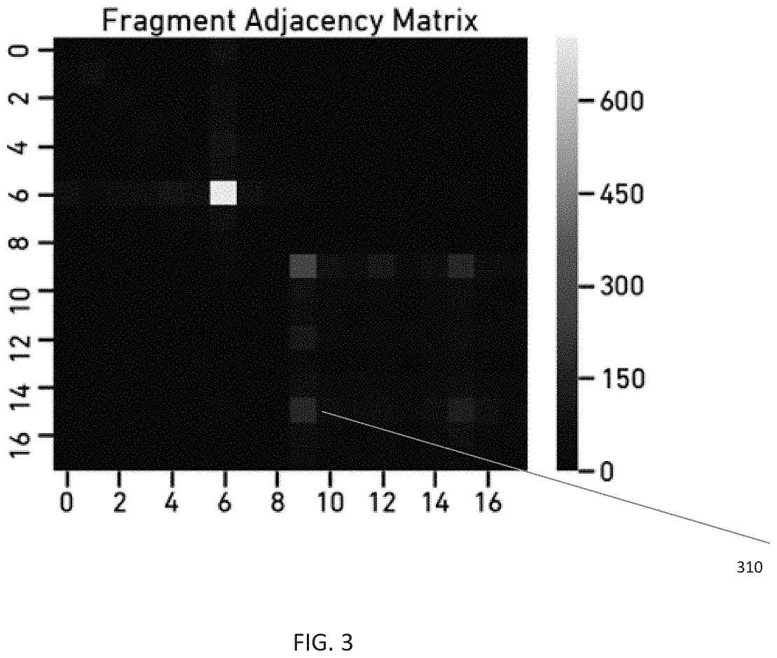

Multiplets can be identified in various ways, one of which is by use of an adjacency matrix such as that illustrated in FIG. 3.

With adjacency matrices, sets of candidate barcodes are identified that may contain pairs of multiplet barcodes 310. A grid showing each barcode on both the X and Y axes is generated. Then, values are filled into the matrix to indicate the number of adjacent fragments between every pair of barcodes.

Typically, a 0 indicates no edge and a 1 indicates an edge. To apply these matrices to analyze for multiplets generally, one would count pairs of adjacent fragments identified through the adjacency matrix, and then keep track of how often the pair of barcodes is seen.

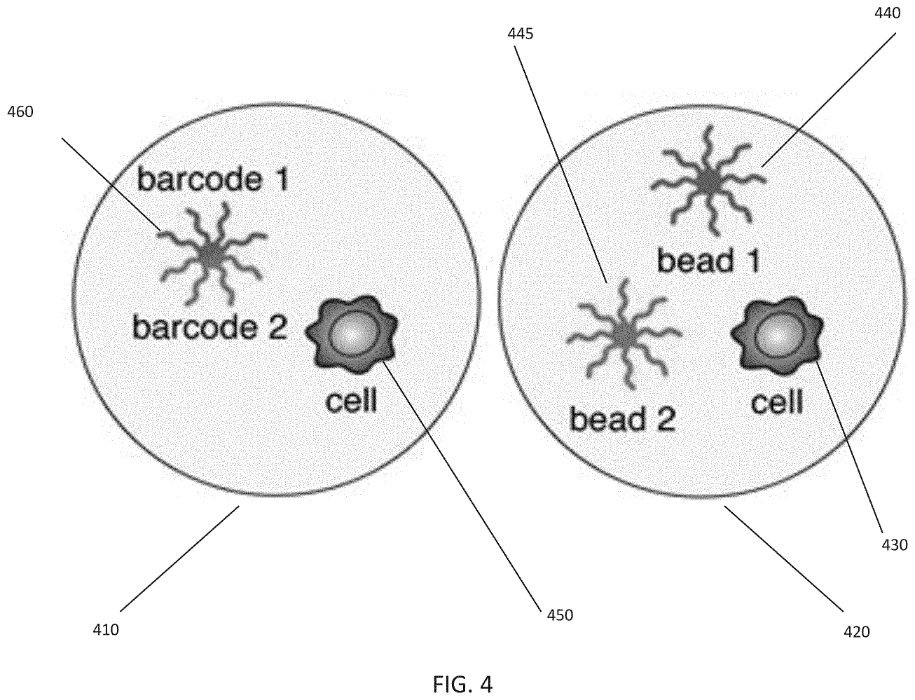

As discussed above, various types of multiplets can be produced as part of the sample preparation process. FIG. 4 illustrates the two example types of multiplets in a partition (e.g., a GEM), partition 410 with barcode multiplets and partition 420 with gel-bead multiplets.

As discussed above, partitions with gel bead multiplets can arise where a cell shares more than one barcoded gel bead in a GEM. In various embodiments, such multiplets are observed to be predominantly doublets where a cell shares two barcoded get beads. This is illustrated in FIG. 4 where partition 420 includes a cell 430 and a first bead 440 and second bead 445. These cells can be manifested as multiple barcodes of the same cell type in the dataset. These beads, and their associated barcodes can be referred to as a minor-major pair of barcodes. For reference, in the case of partition 420, for example, first bead 440 can be referred to as the minor barcode and second bead 445 the major barcode. The presence of these few extra barcodes presumably does not affect the many types of post-sequencing analyses, though even a minor presence can potentially inflate abundance measurements of very rare cell types. As such, these a need does still exist to identify and resolve the handling of these gel bead multiplets.

In accordance with various embodiments herein, a minor-major pair of barcodes (B1, B2) that are part of a putative gel bead doublet can be identified. In various embodiments, the minor-major pair of barcodes can be identified if the pair of barcodes shares more adjoining “linked” fragments (i.e., fragments sharing a transposition event) with each other (B1-B2) as compared to themselves (B1-B1 or B2-B2). The minor barcode can be identified as the one with fewer fragments and can be discarded from the set of total barcodes used in a subsequent cell calling (and counting) process.

One can identify gel-bead multiplets and blacklist the lower count barcode of the pair from subsequent cell calling. Generally, gel-bead multiplets randomly split signal approximately evenly between the two barcodes. Referring to FIG. 3, a processor could build the matrix only for barcodes with a minimum number of total counts. The processor could then look for pairs of barcodes that are mutually nearest neighbors, i.e., where Barcode 1 sees most of its adjacency links with Barcode 2 and vice versa. Applied to the illustrated matrix, gel-bead multiplet 310 is observed where there is a strong cross-signal between barcodes 9 and 15. This pair would then be annotated as a gel-bead multiplet, the lowest count barcode of the par being blacklisted from cell calling.

Barcode multiplets, on the other hand, can occur when a cell associated gel bead is not monoclonal and has the presence of more than one barcode. This is also illustrated in FIG. 4 where partition 410 includes a cell 450 and a gel bead 460 possessing multiple barcodes (e.g., two barcodes as illustrated in FIG. 4).

Accordingly, in accordance with various embodiments herein, the barcodes associated with such multiplets can be identified as the ones sharing significant number of linked fragments with each other as well as having a common suffix or a prefix nucleotide sequence. The “minor” barcode participating in these multiplets can then be masked out while retaining the major barcode as the sole representative of the associated cell of the various embodiments herein.

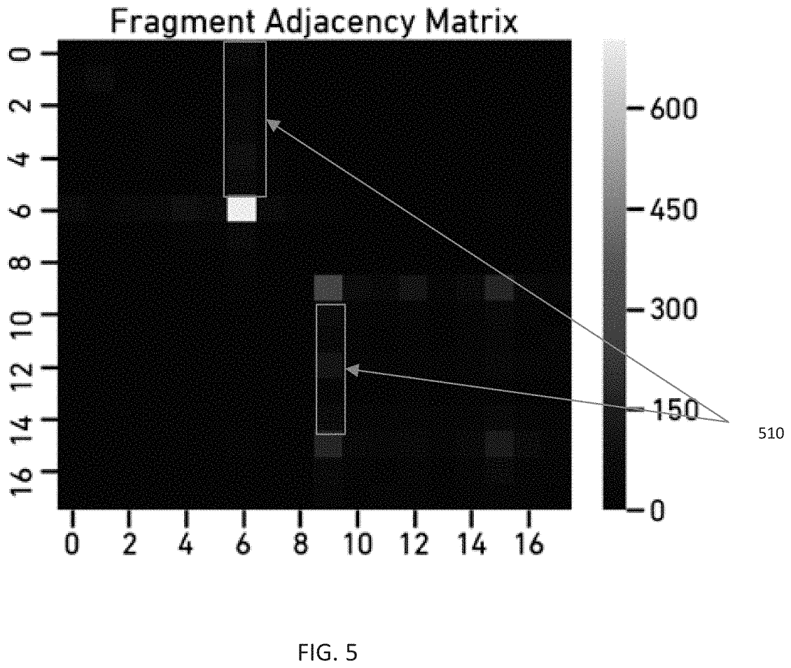

Generally, barcode multiplets produce a dominant barcode and multiple less common contaminant barcodes. Referring now to FIG. 5, a processor could build the matrix that shares a common part A or part B sequence and a common linker. Due to manufacturing methods, these are the only barcodes where plate cross-contamination could cause mixed gel beads. Barcodes are then identified where more cross-signal with some other barcode than self-signal is present. These are represented by boxes 510. These can be annotated as contaminant barcodes and blacklisted them from cell calling.

With the correction of gel bead artifacts and the major barcode retained as the sole representative of the associated cell, cell calling can be performed on the remainder barcodes, in accordance with various embodiments. In various embodiments, a depth-dependent fixed count can be subtracted from all barcode counts to model a whitelist contamination (free barcodes present in solution along with the gel beads). This fixed count can be understood to be the estimated number of fragments per barcode that originated from a different barcoded bead (e.g., a Gel bead-In EMulsion (GEM)), when assuming a contamination rate of 0.02. A mixture model of two negative binomial distributions can then be fit to capture the signal and noise. Setting an odds ratio of 100000, which appeared to work best with internal testing in accordance with various embodiments herein, the barcodes that correspond to real cells can be separated from the non-cell barcodes (also referred to as low targeting barcodes). It is understood that other odds-ratios in addition to the ones disclosed herein can be selected in accordance with various embodiments herein.

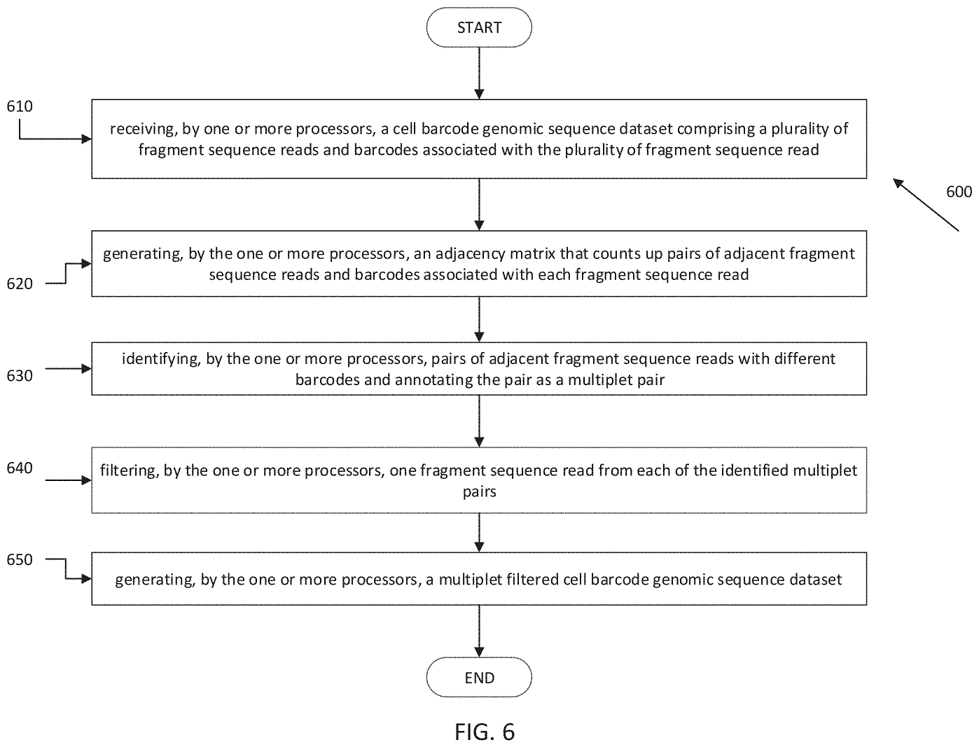

FIG. 6 is an exemplary flowchart showing a method 600 for filtering open chromatin regions on a cell barcode genomic sequence dataset, in accordance with various embodiments.

In step 610, one or more processors receive a cell barcode genomic sequence dataset comprising a plurality of fragment sequence reads and barcodes associated with the plurality of fragment sequence read.

In step 620, the one or more processors generate an adjacency matrix that counts up pairs of adjacent fragment sequence reads and barcodes associated with each fragment sequence read.

In step 630, one or more processors identify pairs of adjacent fragment sequence reads with different barcodes and annotating the pair as a multiplet pair.

In step 640, one or more processors filter one fragment sequence read from each of the identified multiplet pairs.

In step 650, one or more processors generate a multiplet filtered cell barcode genomic sequence dataset.

In various embodiments, one or more processors can identify a pair of adjacent fragment sequence reads as a multiplet when each member of the pair of adjacent fragment sequence reads are found more often with different barcodes than with a same barcode. Further, one or more processors can filter the fragment sequence read from each multiplet pair based on an associated barcode having the lowest count in the adjacency matrix. Alternatively, one or more processors can filter the fragment sequence read from each multiplet pair based on an associated barcode having more cross signal with another barcode than with the same associated barcode. Further, the adjacency matrix can be constructed only for pairs of barcodes that share a common sequence.

In various embodiments, the method 600 can further include the step of identifying and removing low targeting barcodes.

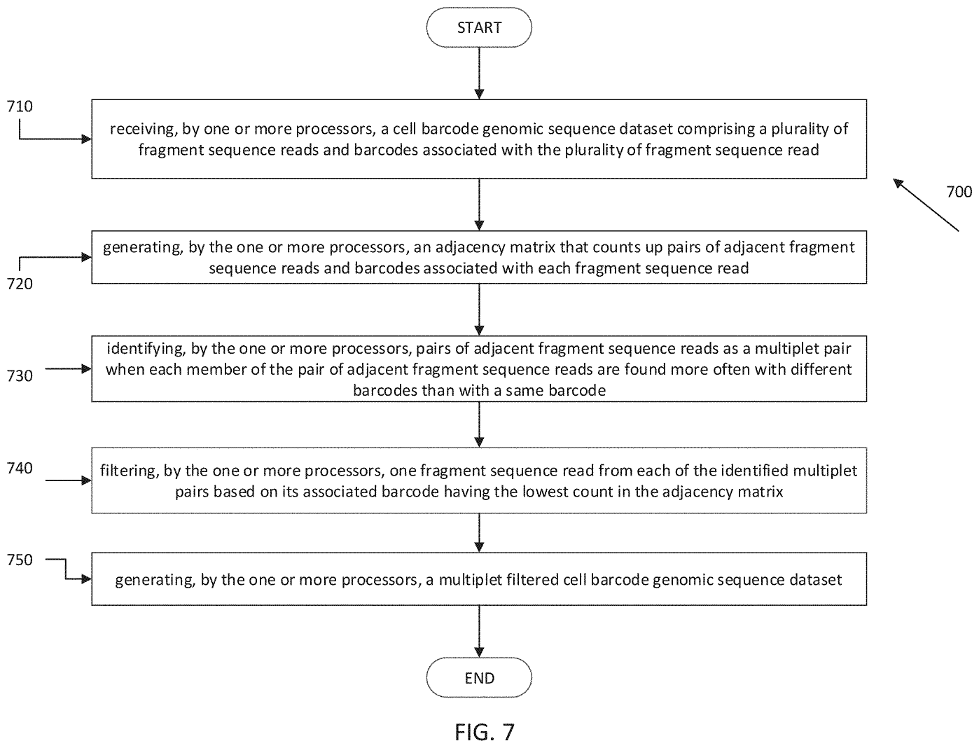

FIG. 7 is an exemplary flowchart showing a method 700 for filtering open chromatin regions on a cell barcode genomic sequence dataset, in accordance with various embodiments.

In step 710, one or more processors receive a cell barcode genomic sequence dataset comprising a plurality of fragment sequence reads and barcodes associated with the plurality of fragment sequence read.

In step 720, the one or more processors generate an adjacency matrix that counts up pairs of adjacent fragment sequence reads and barcodes associated with each fragment sequence read. In step 730, one or more processors identify pairs of adjacent fragment sequence reads as a multiplet pair when each member of the pair of adjacent fragment sequence reads are found more often with different barcodes than with a same barcode.

In step 740, one or more processors filter one fragment sequence read from each of the identified multiplet pairs based on its associated barcode having the lowest count in the adjacency matrix.

In step 750, one or more processors generate a multiplet filtered cell barcode genomic sequence dataset.

In various embodiments, one or more processors can identify a pair of adjacent fragment sequence reads as a multiplet when each member of the pair of adjacent fragment sequence reads are found more often with different barcodes than with a same barcode. Further, in some embodiments, one or more processors can filter the fragment sequence read from each multiplet pair based on an associated barcode having more cross signal with another barcode than with the same associated barcode. Further, the adjacency matrix can be constructed only for pairs of barcodes that share a common sequence.

in various embodiments, the method 700 can further include the step of identifying and removing low targeting barcodes.

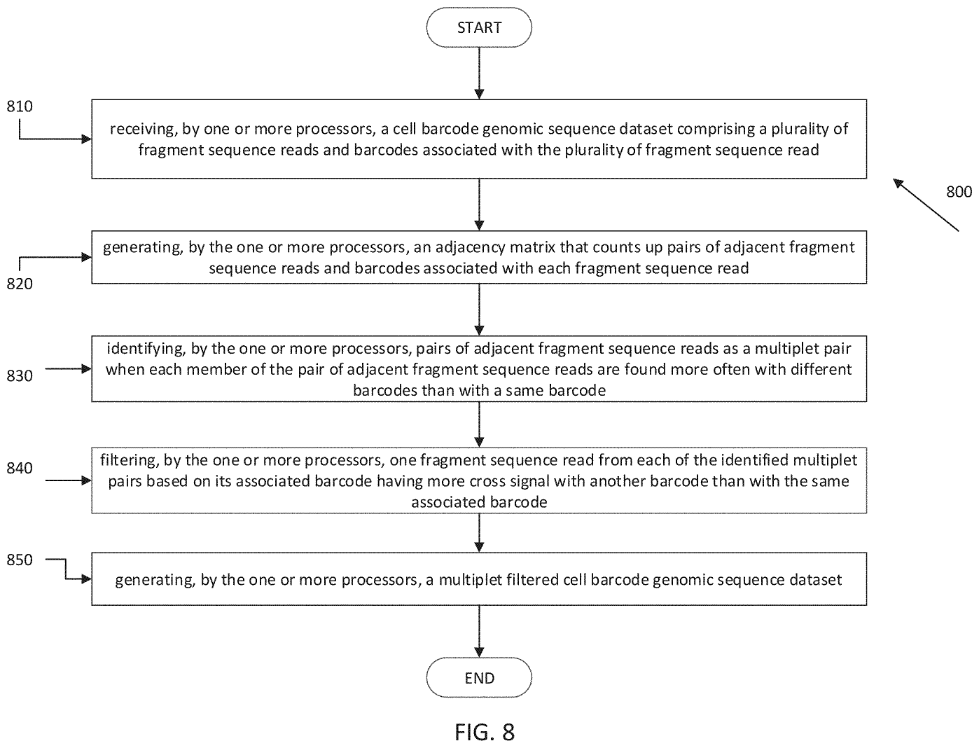

FIG. 8 is an exemplary flowchart showing a method 800 for filtering open chromatin regions on a cell barcode genomic sequence dataset, in accordance with various embodiments.

In step 810, one or more processors receive a cell barcode genomic sequence dataset comprising a plurality of fragment sequence reads and barcodes associated with the plurality of fragment sequence read.

In step 820, the one or more processors generate an adjacency matrix that counts up pairs of adjacent fragment sequence reads and barcodes associated with each fragment sequence read. In various embodiments, the adjacency matrix can be constructed pairs of barcodes that share a common sequence.

In step 830, one or more processors identify pairs of adjacent fragment sequence reads as a multiplet pair when each member of the pair of adjacent fragment sequence reads are found more often with different barcodes than with a same barcode.

In step 840, one or more processors filter one fragment sequence read from each of the identified multiplet pairs based on its associated barcode having more cross signal with another barcode than with the same associated barcode.

In step 850, one or more processors generate a multiplet filtered cell barcode genomic sequence dataset.

In various embodiments, one or more processors can identify a pair of adjacent fragment sequence reads as a multiplet when each member of the pair of adjacent fragment sequence reads are found more often with different barcodes than with a same barcode. Further, one or more processors can filter the fragment sequence read from each multiplet pair based on an associated barcode having the lowest count in the adjacency matrix.

In various embodiments, the method 800 can further include the step of identifying and removing low targeting barcodes.

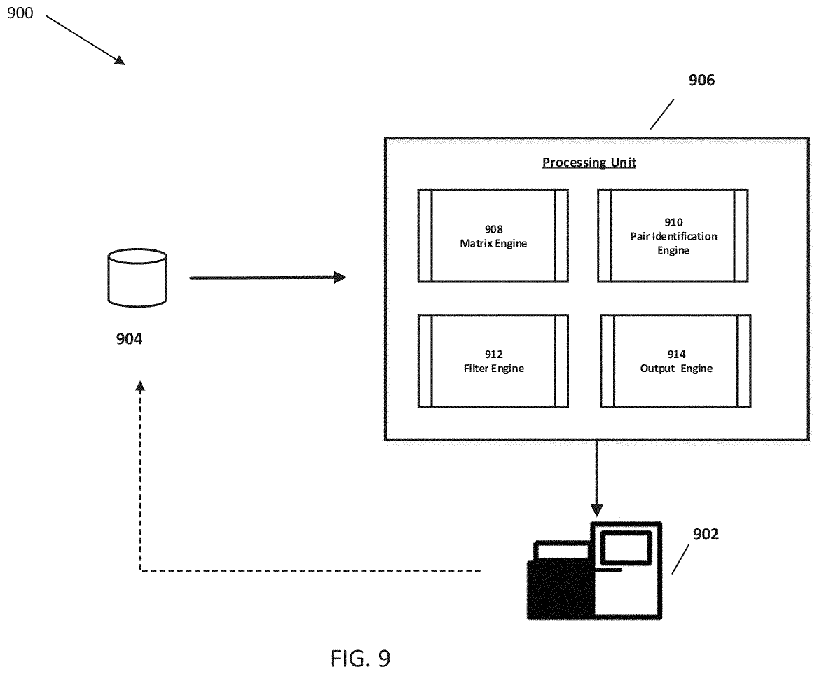

FIG. 9 is a schematic diagram of an example system 900 for filtering open chromatin regions on a cell barcode genomic sequence dataset, in accordance with various embodiments. The system 900 can include a display 902, a data source 904, and a computing device such as a processing unit 906. The processing unit 906 can include a matrix engine 908, pair identification engine 910, a filter engine 912, and an output engine 914.

In some embodiments, the processing unit 906 can be configured to receive a cell barcode genomic sequence dataset comprising a plurality of fragment sequence reads and barcodes associated with the plurality of fragment sequence reads from the data source 904. In various embodiments, the matrix engine 908 can be configured to generate an adjacency matrix that counts up pairs of adjacent fragment sequence reads and barcodes associated with each fragment sequence read. The pair identification engine 910 can be configured to identify pairs of adjacent fragment sequence reads with different barcodes and annotating the pair as a multiplet pair. The filter engine 912 can be configured to filter one fragment sequence read from each of the identified multiplet pairs. The output engine 914 can be configured to generate a multiplet filtered cell barcode genomic sequence dataset as an output. The processing unit 906 can be further configured to send the output to the display 902. In various embodiments, the data source 904 can be configured to receive a cell barcode genomic sequence dataset comprising a plurality of fragment sequence reads and barcodes associated with the plurality of fragment sequence reads from a display such as the display 902.

In some other embodiments, the processing unit 906 can be configured to receive a cell barcode genomic sequence dataset comprising a plurality of fragment sequence reads and barcodes associated with the plurality of fragment sequence reads from the data source 904. In various embodiments, the matrix engine 908 can be configured to generate an adjacency matrix that counts up pairs of adjacent fragment sequence reads and barcodes associated with each fragment sequence read. The pair identification engine 910 can be configured to identify pairs of adjacent fragment sequence reads as a multiplet pair when each member of the pair of adjacent fragment sequence reads are found more often with different barcodes than with a same barcode. The filter engine 912 can be configured to filter one fragment sequence read from each of the identified multiplet pairs based on its associated barcode having a lowest count in the adjacency matrix. The output engine 914 can be configured to generate a multiplet filtered cell barcode genomic sequence dataset as an output. The processing unit 906 can be further configured to send the output to the display 902. In various embodiments, the data source 904 can be configured to receive a cell barcode genomic sequence dataset comprising a plurality of fragment sequence reads and barcodes associated with the plurality of fragment sequence reads from a display such as the display 902.

In some other embodiments, the processing unit 906 can be configured to receive a cell barcode genomic sequence dataset comprising a plurality of fragment sequence reads and barcodes associated with the plurality of fragment sequence reads form the data source 904. In accordance with various embodiments, the matrix engine 908 can be configured to generate an adjacency matrix that counts up pairs of adjacent fragment sequence reads and barcodes associated with each fragment sequence read. The pair identification engine 910 can be configured to identify pairs of adjacent fragment sequence reads as a multiplet pair when each member of the pair of adjacent fragment sequence reads are found more often with different barcodes than with a same barcode. The filter engine 912 can be configured to filter one fragment sequence read from each of the identified multiplet pairs based on its associated barcode having more cross signal with another barcode than with the same associated barcode. The output engine 914 can be configured to generate a multiplet filtered cell barcode genomic sequence dataset as an output. The processing unit 906 can be further configured to send the output to the display 902. In various embodiments, the data source 904 can be configured to receive a cell barcode genomic sequence dataset comprising a plurality of fragment sequence reads and barcodes associated with the plurality of fragment sequence reads from a display such as the display 902.

### Computer System

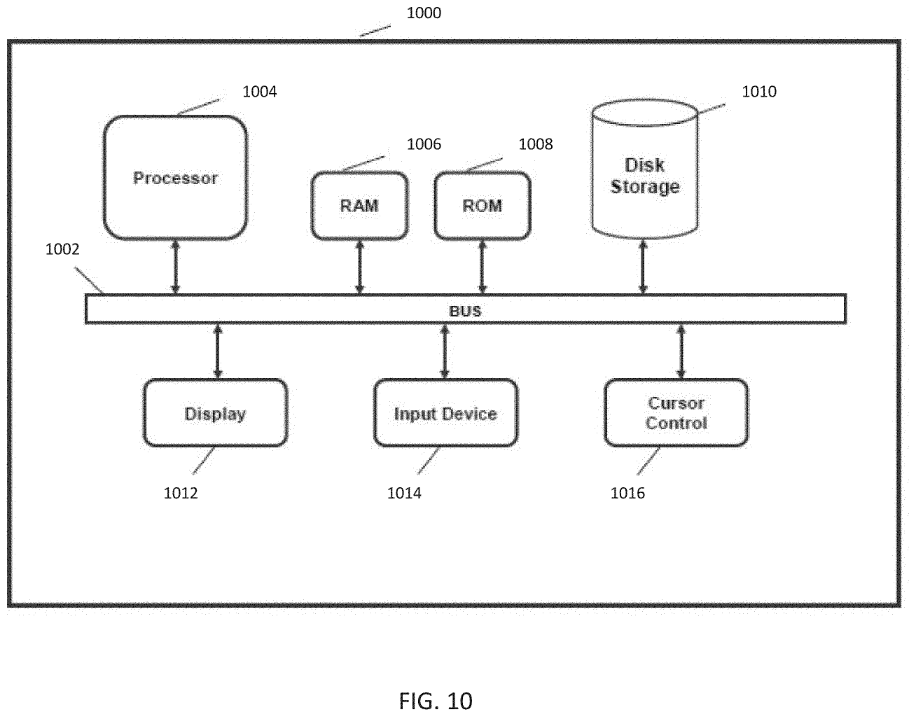

In accordance with various embodiments, the methods for filtering open chromatin regions on a cell barcode genomic sequence dataset, such as the example methods 600/700/800 illustrated in FIGS. 6, 7 and 8, can be implemented via computer software or hardware. Similarly, systems for filtering open chromatin regions on a cell barcode genomic sequence dataset, such as the example system 900, can be implemented via computer software or hardware. That is, the methods and systems disclosed herein can be implemented using a computer system 1000 of FIG. 10 via, for example, non-transitory computer-readable medium storing computer instructions for filtering open chromatin regions on a cell barcode genomic sequence dataset.

FIG. 10 is a block diagram that illustrates the computer system 1000, upon which embodiments of the present teachings may be implemented. In various embodiments of the present teachings, computer system 1000 can include a bus 1002 or other communication mechanism for communicating information, and a processor 1004 coupled with bus 1002 for processing information. In various embodiments, computer system 1000 can also include a memory, which can be a random-access memory (RAM) 1006 or other dynamic storage device, coupled to bus 1002 for determining instructions to be executed by processor 1004. Memory also can be used for storing temporary variables or other intermediate information during execution of instructions to be executed by processor 1004. In various embodiments, computer system 1000 can further include a read only memory (ROM) 1008 or other static storage device coupled to bus 1002 for storing static information and instructions for processor 1004. A storage device 1010, such as a magnetic disk or optical disk, can be provided and coupled to bus 1002 for storing information and instructions.

In various embodiments, computer system 1000 can be coupled via bus 1002 to a display 1012, such as a cathode ray tube (CRT) or liquid crystal display (LCD), for displaying information to a computer user. An input device 1014, including alphanumeric and other keys, can be coupled to bus 1002 for communicating information and command selections to processor 1004. Another type of user input device is a cursor control 1016, such as a mouse, a trackball or cursor direction keys for communicating direction information and command selections to processor 1004 and for controlling cursor movement on display 1012. This input device 1014 typically has two degrees of freedom in two axes, a first axis (i.e., x) and a second axis (i.e., y), that allows the device to specify positions in a plane. However, it should be understood that input devices 1014 allowing for three-dimensional (x, y and z) cursor movement are also contemplated herein.

Consistent with certain implementations of the present teachings, results can be provided by computer system 1000 in response to processor 1004 executing one or more sequences of one or more instructions contained in memory 1006. Such instructions can be read into memory 1006 from another computer-readable medium or computer-readable storage medium, such as storage device 1010. Execution of the sequences of instructions contained in memory 1006 can cause processor 1004 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 1004 for execution. Such a medium can take many forms, including but not limited to, non-volatile media, volatile media, and transmission media. Examples of non-volatile media can include, but are not limited to, optical, solid state, magnetic disks, such as storage device 1010. Examples of volatile media can include, but are not limited to, dynamic memory, such as memory 1006. Examples of transmission media can include, but are not limited to, coaxial cables, copper wire, and fiber optics, including the wires that comprise bus 1002.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 1004 of computer system 1000 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein flow charts, diagrams and accompanying disclosure can be implemented using computer system 1000 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 1000 of FIG. 10, whereby processor 1004 would execute the analyses and determinations provided by these engines, subject to instructions provided by any one of, or a combination of, memory components 1006/1008/1010 and user input provided via input device 1014.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

### Recitation of Embodiments

Embodiment 1. A method for filtering open chromatin regions on a cell barcode genomic sequence dataset, comprising: receiving, by one or more processors, a cell barcode genomic sequence dataset comprising a plurality of fragment sequence reads and barcodes associated with the plurality of fragment sequence reads; generating, by the one or more processors, an adjacency matrix that counts up pairs of adjacent fragment sequence reads and barcodes associated with each fragment sequence read; identifying, by the one or more processors, pairs of adjacent fragment sequence reads with different barcodes and annotating the pair as a multiplet pair; filtering, by the one or more processors, one fragment sequence read from each of the identified multiplet pairs; and generating, by the one or more processors, a multiplet filtered cell barcode genomic sequence dataset.

Embodiment 2. The method of Embodiment 1, wherein a pair of adjacent fragment sequence reads are identified as a multiplet when each member of the pair of adjacent fragment sequence reads are found more often with different barcodes than with a same barcode, and wherein the fragment sequence read filtered from each multiplet pair is selected based on an associated barcode having a lowest count in the adjacency matrix.

Embodiment 3. The method of Embodiment 1, wherein a pair of adjacent fragment sequence reads are identified as a multiplet when each member of the pair of adjacent fragment sequence reads are found more often with different barcodes than with a same barcode, and wherein the fragment sequence read filtered from each multiplet pair is selected based on an associated barcode having more cross signal with another barcode than with a same associated barcode.

Embodiment 4. The method of Embodiment 1, wherein the adjacency matrix is constructed only for pairs of barcodes that share a common sequence.

Embodiment 5. The method of Embodiment 1, further comprising identifying and removing low targeting barcodes.

Embodiment 6. A non-transitory computer-readable medium storing computer instructions for filtering open chromatin regions on a cell barcode genomic sequence dataset, the computer instructions comprising: receiving, by one or more processors, a cell barcode genomic sequence dataset comprising a plurality of fragment sequence reads and barcodes associated with the plurality of fragment sequence reads; generating, by the one or more processors, an adjacency matrix that counts up pairs of adjacent fragment sequence reads and barcodes associated with each fragment sequence read; identifying, by the one or more processors, pairs of adjacent fragment sequence reads with different barcodes and annotating the pair as a multiplet pair; filtering, by the one or more processors, one fragment sequence read from each of the identified multiplet pairs; and generating, by the one or more processors, a multiplet filtered cell barcode genomic sequence dataset.

Embodiment 7. The non-transitory computer-readable medium of Embodiment 6, wherein a pair of adjacent fragment sequence reads are identified as a multiplet when each member of the pair of adjacent fragment sequence reads are found more often with different barcodes than with a same barcode, and wherein the fragment sequence read filtered from each multiplet pair is selected based on an associated barcode having a lowest count in the adjacency matrix.

Embodiment 8. The non-transitory computer-readable medium of Embodiment 6, wherein a pair of adjacent fragment sequence reads are identified as a multiplet when each member of the pair of adjacent fragment sequence reads are found more often with different barcodes than with a same barcode, and wherein the fragment sequence read filtered from each multiplet pair is selected based on an associated barcode having more cross signal with another barcode than with a same associated barcode.

Embodiment 9. The non-transitory computer-readable medium of Embodiment 6, wherein the adjacency matrix is constructed only for pairs of barcodes that share a common sequence.

Embodiment 10. The non-transitory computer-readable medium of Embodiment 6, wherein the computer instructions further comprises identifying and removing low targeting barcodes.

Embodiment 11. A system for filtering open chromatin regions on a cell barcode genomic sequence dataset, comprising: a data source for receiving, by one or more processors, a cell barcode genomic sequence dataset comprising a plurality of fragment sequence reads and barcodes associated with the plurality of fragment sequence reads; a computing device communicatively connected to the data source and comprises: a matrix engine configured to generate an adjacency matrix that counts up pairs of adjacent fragment sequence reads and barcodes associated with each fragment sequence read; a pair identification engine configured to identify pairs of adjacent fragment sequence reads with different barcodes and annotating the pair as a multiplet pair; a filter engine configured to filter one fragment sequence read from each of the identified multiplet pairs; and an output engine configured to generate a multiplet filtered cell barcode genomic sequence dataset.

Embodiment 12. The system of Embodiment 11, wherein a pair of adjacent fragment sequence reads are identified as a multiplet when each member of the pair of adjacent fragment sequence reads are found more often with different barcodes than with a same barcode, and wherein the fragment sequence read filtered from each multiplet pair is selected based on an associated barcode having a lowest count in the adjacency matrix.

Embodiment 13. The system of Embodiment 11, wherein a pair of adjacent fragment sequence reads are identified as a multiplet when each member of the pair of adjacent fragment sequence reads are found more often with different barcodes than with a same barcode, and wherein the fragment sequence read filtered from each multiplet pair is selected based on an associated barcode having more cross signal with another barcode than with a same associated barcode.

Embodiment 14. The system of Embodiment 11, wherein the adjacency matrix is constructed only for pairs of barcodes that share a common sequence.

Embodiment 15. The system of Embodiment 11, wherein the computing device is further configured to identify and remove low targeting barcodes.

Embodiment 16. A method for filtering open chromatin regions on a cell barcode genomic sequence dataset, comprising: receiving, by one or more processors, a cell barcode genomic sequence dataset comprising a plurality of fragment sequence reads and barcodes associated with the plurality of fragment sequence reads; generating, by the one or more processors, an adjacency matrix that counts up pairs of adjacent fragment sequence reads and barcodes associated with each fragment sequence read; identifying, by the one or more processors, pairs of adjacent fragment sequence reads as a multiplet pair when each member of the pair of adjacent fragment sequence reads are found more often with different barcodes than with a same barcode; filtering, by the one or more processors, one fragment sequence read from each of the identified multiplet pairs based on its associated barcode having a lowest count in the adjacency matrix; and generating, by the one or more processors, a multiplet filtered cell barcode genomic sequence dataset.

Embodiment 17. The method of Embodiment 16, wherein a pair of adjacent fragment sequence reads are identified as a multiplet when each member of the pair of adjacent fragment sequence reads are found more often with different barcodes than with a same barcode, and wherein the fragment sequence read filtered from each multiplet pair is selected based on an associated barcode having more cross signal with another barcode than with the same associated barcode.

Embodiment 18. The method of Embodiment 16, wherein the adjacency matrix is constructed only for pairs of barcodes that share a common sequence.

Embodiment 19. The method of Embodiment 16, further comprising identifying and removing low targeting barcodes.

Embodiment 20. A non-transitory computer-readable medium storing computer instructions for filtering open chromatin regions on a cell barcode genomic sequence dataset, comprising: receiving, by one or more processors, a cell barcode genomic sequence dataset comprising a plurality of fragment sequence reads and barcodes associated with the plurality of fragment sequence reads; generating, by the one or more processors, an adjacency matrix that counts up pairs of adjacent fragment sequence reads and barcodes associated with each fragment sequence read; identifying, by the one or more processors, pairs of adjacent fragment sequence reads as a multiplet pair when each member of the pair of adjacent fragment sequence reads are found more often with different barcodes than with a same barcode; filtering, by the one or more processors, one fragment sequence read from each of the identified multiplet pairs based on its associated barcode having a lowest count in the adjacency matrix; and generating, by the one or more processors, a multiplet filtered cell barcode genomic sequence dataset.

Embodiment 21. The non-transitory computer-readable medium of Embodiment 20, wherein a pair of adjacent fragment sequence reads are identified as a multiplet when each member of the pair of adjacent fragment sequence reads are found more often with different barcodes than with a same barcode, and wherein the fragment sequence read filtered from each multiplet pair is selected based on an associated barcode having more cross signal with another barcode than with the same associated barcode.

Embodiment 22. The non-transitory computer-readable medium of Embodiment 20, wherein the adjacency matrix is constructed only for pairs of barcodes that share a common sequence.

Embodiment 23. The non-transitory computer-readable medium of Embodiment 20, wherein the computing instructions further comprise identifying and removing low targeting barcodes.

Embodiment 24. A system for filtering open chromatin regions on a cell barcode genomic sequence dataset, comprising: a data source for receiving, by one or more processors, a cell barcode genomic sequence dataset comprising a plurality of fragment sequence reads and barcodes associated with the plurality of fragment sequence reads; a computing device communicatively connected to the data source and comprises: a matrix engine configured to generate an adjacency matrix that counts up pairs of adjacent fragment sequence reads and barcodes associated with each fragment sequence read; a pair identification engine configured to identify pairs of adjacent fragment sequence reads as a multiplet pair when each member of the pair of adjacent fragment sequence reads are found more often with different barcodes than with a same barcode; a filter engine configured to filter one fragment sequence read from each of the identified multiplet pairs based on its associated barcode having a lowest count in the adjacency matrix; and an output engine configured to generate a multiplet filtered cell barcode genomic sequence dataset.

Embodiment 25. The system of Embodiment 24, wherein a pair of adjacent fragment sequence reads are identified as a multiplet when each member of the pair of adjacent fragment sequence reads are found more often with different barcodes than with a same barcode, and wherein the fragment sequence read filtered from each multiplet pair is selected based on an associated barcode having more cross signal with another barcode than with the same associated barcode.

Embodiment 26. The system of Embodiment 24, wherein the adjacency matrix is constructed only for pairs of barcodes that share a common sequence.

Embodiment 27. The system of Embodiment 24, wherein the computing device is further configured to identify and remove low targeting barcodes.

Embodiment 28. A method for filtering open chromatin regions on a cell barcode genomic sequence dataset, comprising: receiving, by one or more processors, a cell barcode genomic sequence dataset comprising a plurality of fragment sequence reads and barcodes associated with the plurality of fragment sequence reads; generating, by the one or more processors, an adjacency matrix that counts up pairs of adjacent fragment sequence reads and barcodes associated with each fragment sequence read; identifying, by the one or more processors, pairs of adjacent fragment sequence reads as a multiplet pair when each member of the pair of adjacent fragment sequence reads are found more often with different barcodes than with a same barcode; filtering, by the one or more processors, one fragment sequence read from each of the identified multiplet pairs based on its associated barcode having more cross signal with another barcode than with the same associated barcode; and generating, by the one or more processors, a multiplet filtered cell barcode genomic sequence dataset.

Embodiment 29. The method of Embodiment 28, wherein the adjacency matrix is constructed only for pairs of barcodes that share a common sequence.

Embodiment 30. The method of Embodiment 28, wherein a pair of adjacent fragment sequence reads are identified as a multiplet when each member of the pair of adjacent fragment sequence reads are found more often with different barcodes than with a same barcode, and wherein the fragment sequence read filtered from each multiplet pair is selected based on an associated barcode having a lowest count in the adjacency matrix.

Embodiment 31. The method of Embodiment 28, further comprising identifying and removing low targeting barcodes.

Embodiment 32. A non-transitory computer-readable medium storing computer instructions for filtering open chromatin regions on a cell barcode genomic sequence dataset, the computer instructions comprising: receiving, by one or more processors, a cell barcode genomic sequence dataset comprising a plurality of fragment sequence reads and barcodes associated with the plurality of fragment sequence reads; generating, by the one or more processors, an adjacency matrix that counts up pairs of adjacent fragment sequence reads and barcodes associated with each fragment sequence read; identifying, by the one or more processors, pairs of adjacent fragment sequence reads as a multiplet pair when each member of the pair of adjacent fragment sequence reads are found more often with different barcodes than with a same barcode; filtering, by the one or more processors, one fragment sequence read from each of the identified multiplet pairs based on its associated barcode having more cross signal with another barcode than with the same associated barcode; and generating, by the one or more processors, a multiplet filtered cell barcode genomic sequence dataset.

Embodiment 33. The non-transitory computer-readable medium of Embodiment 32, wherein the adjacency matrix is constructed only for pairs of barcodes that share a common sequence.

Embodiment 34. The non-transitory computer-readable medium of Embodiment 32, wherein a pair of adjacent fragment sequence reads are identified as a multiplet when each member of the pair of adjacent fragment sequence reads are found more often with different barcodes than with a same barcode, and wherein the fragment sequence read filtered from each multiplet pair is selected based on an associated barcode having a lowest count in the adjacency matrix.

Embodiment 35. The non-transitory computer-readable medium of Embodiment 32, wherein the computer instructions further comprises identifying and removing low targeting barcodes.

Embodiment 36. A system for filtering open chromatin regions on a cell barcode genomic sequence dataset, comprising: a data source for receiving, by one or more processors, a cell barcode genomic sequence dataset comprising a plurality of fragment sequence reads and barcodes associated with the plurality of fragment sequence reads; a computing device communicatively connected to the data source and comprises: a matrix engine configured to generate an adjacency matrix that counts up pairs of adjacent fragment sequence reads and barcodes associated with each fragment sequence read; a pair identification engine configured to pairs of adjacent fragment sequence reads as a multiplet pair when each member of the pair of adjacent fragment sequence reads are found more often with different barcodes than with a same barcode; a filter engine configured to filter one fragment sequence read from each of the identified multiplet pairs based on its associated barcode having more cross signal with another barcode than with the same associated barcode; and an output engine configured to generate a multiplet filtered cell barcode genomic sequence dataset.

Embodiment 37. The system of Embodiment 36, wherein the adjacency matrix is constructed only for pairs of barcodes that share a common sequence.

Embodiment 38. The system of Embodiment 36, wherein a pair of adjacent fragment sequence reads are identified as a multiplet when each member of the pair of adjacent fragment sequence reads are found more often with different barcodes than with a same barcode, and wherein the fragment sequence read filtered from each multiplet pair is selected based on an associated barcode having a lowest count in the adjacency matrix.

Embodiment 39. The system of Embodiment 36, wherein the computing device is configured to identify and remove low targeting barcodes.

What is claimed is:

1. A method for filtering open chromatin regions on a cell barcode genomic sequence dataset, comprising:
receiving, by one or more processors, a cell barcode genomic sequence dataset comprising a plurality of fragment sequence reads and barcodes associated with the plurality of fragment sequence reads;
generating, by the one or more processors, an adjacency matrix that counts up pairs of adjacent fragment sequence reads and barcodes associated with each fragment sequence read;
identifying, by the one or more processors, pairs of adjacent fragment sequence reads with different barcodes and annotating the pair as a multiplet pair;
filtering, by the one or more processors, one fragment sequence read from each of the identified multiplet pairs; and
generating, by the one or more processors, a multiplet filtered cell barcode genomic sequence dataset.

2. The method of claim 1, wherein a pair of adjacent fragment sequence reads are identified as a multiplet when each member of the pair of adjacent fragment sequence reads are found more often with different barcodes than with a same barcode.

3. The method of claim 2, wherein the fragment sequence read filtered from each multiplet pair is selected based on an associated barcode having a lowest count in the adjacency matrix.

4. The method of claim 1, wherein a pair of adjacent fragment sequence reads are identified as a multiplet when each member of the pair of adjacent fragment sequence reads are found more often with different barcodes than with a same barcode.

5. The method of claim 4, wherein the fragment sequence read filtered from each multiplet pair is selected based on an associated barcode having more cross signal with another barcode than with a same associated barcode.

6. The method of claim 1, wherein the adjacency matrix is constructed only for pairs of barcodes that share a common sequence.

7. The method of claim 1, further comprising identifying and removing low targeting barcodes.

8. A non-transitory computer-readable medium storing computer instructions for filtering open chromatin regions on a cell barcode genomic sequence dataset, the computer instructions comprising:
receiving, by one or more processors, a cell barcode genomic sequence dataset comprising a plurality of fragment sequence reads and barcodes associated with the plurality of fragment sequence reads;
generating, by the one or more processors, an adjacency matrix that counts up pairs of adjacent fragment sequence reads and barcodes associated with each fragment sequence read;
identifying, by the one or more processors, pairs of adjacent fragment sequence reads with different barcodes and annotating the pair as a multiplet pair;
filtering, by the one or more processors, one fragment sequence read from each of the identified multiplet pairs; and
generating, by the one or more processors, a multiplet filtered cell barcode genomic sequence dataset.

9. The non-transitory computer-readable medium of claim 8, wherein a pair of adjacent fragment sequence reads are identified as a multiplet when each member of the pair of adjacent fragment sequence reads are found more often with different barcodes than with a same barcode.

10. The non-transitory computer-readable medium of claim 9, wherein the fragment sequence read filtered from each multiplet pair is selected based on an associated barcode having a lowest count in the adjacency matrix.

11. The non-transitory computer-readable medium of claim 8, wherein a pair of adjacent fragment sequence reads are identified as a multiplet when each member of the pair of adjacent fragment sequence reads are found more often with different barcodes than with a same barcode.

12. The non-transitory computer-readable medium of claim 11, wherein the fragment sequence read filtered from each multiplet pair is selected based on an associated barcode having more cross signal with another barcode than with a same associated barcode.

13. The non-transitory computer-readable medium of claim 8, wherein the adjacency matrix is constructed only for pairs of barcodes that share a common sequence.

14. The non-transitory computer-readable medium of claim 8, further comprising identifying and removing low targeting barcodes.

15. A system for filtering open chromatin regions on a cell barcode genomic sequence dataset, comprising:
a data source for receiving, by one or more processors, a cell barcode genomic sequence dataset comprising a plurality of fragment sequence reads and barcodes associated with the plurality of fragment sequence reads;
a computing device communicatively connected to the data source and comprises:
a matrix engine configured to generate an adjacency matrix that counts up pairs of adjacent fragment sequence reads and barcodes associated with each fragment sequence read;
a pair identification engine configured to identify pairs of adjacent fragment sequence reads with different barcodes and annotating the pair as a multiplet pair;
a filter engine configured to filter one fragment sequence read from each of the identified multiplet pairs; and
an output engine configured to generate a multiplet filtered cell barcode genomic sequence dataset.

16. The system of claim 15, wherein a pair of adjacent fragment sequence reads are identified as a multiplet when each member of the pair of adjacent fragment sequence reads are found more often with different barcodes than with a same barcode.

17. The system of claim 16, wherein the fragment sequence read filtered from each multiplet pair is selected based on an associated barcode having a lowest count in the adjacency matrix.

18. The system of claim 15, wherein a pair of adjacent fragment sequence reads are identified as a multiplet when each member of the pair of adjacent fragment sequence reads are found more often with different barcodes than with a same barcode.

19. The system of claim 18, wherein the fragment sequence read filtered from each multiplet pair is selected based on an associated barcode having more cross signal with another barcode than with a same associated barcode.

70. The system of claim 15, wherein the adjacency matrix is constructed only for pairs of barcodes that share a common sequence.
